(19) United States
(12) Patent Application Publication
(10) Pub. No.: US 2022/0365334 A1
(43) Pub. Date: Nov. 17, 2022

(54) ENDOSCOPE AND IMAGING MODULE

(71) Applicant: OLYMPUS CORPORATION, Tokyo (JP)

(72) Inventors: Takatoshi IGARASHI, Kamiina-gun (JP); Satoru ADACHI, Tsuchiura-shi (JP); Nana AKAHANE, Yamanashi-shi (JP); Masashi SAITO, Tokyo (JP)

(73) Assignee: OLYMPUS CORPORATION, Tokyo (JP)

(21) Appl. No.: 17/865,738

(22) Filed: Jul. 15, 2022

Related U.S. Application Data
(63) Continuation of application No. PCT/JP2020/005560, filed on Feb. 13, 2020.

(51) Int. Cl. G02B 23/24; H01L 27/146; H04N 5/369

(52) U.S. Cl. CPC G02B 23/2484; H01L 27/14634; H04N 5/3698; H04N 5/379; H01L 27/14636; H04N 2005/2255

(57) ABSTRACT

An endoscope includes: an image sensor having a light receiving plane; a laminate provided to be opposed to an opposite side of the image sensor from the light receiving plane and having a plurality of layers formed by lamination of a plurality of semiconductor elements; and an insertion section having the image sensor and the laminate therein. The laminate includes: a first active layer in which a first active element is provided; a first passive layer in which a first passive element is provided and which is provided between the first active layer and the image sensor; and a through-silicon via provided in each of the first active layer and the first passive layer.

### CROSS REFERENCE TO RELATED APPLICATION

This application is a continuation of International Patent Application No. PCT/JP2020/005560, having an international filing date of Feb. 13, 2020, which designated the United States, the entirety of which is incorporated herein by reference.

### BACKGROUND

A method of using an imaging module including lamination of semiconductor elements has been known. Using an imaging module in the form of a semiconductor laminate allows for reducing the diameter of a device including the imaging module. For example, WO 2017/073440 discloses an imaging module having a semiconductor laminate provided on a back side of an image sensor, and an endoscope including the imaging module. Using an imaging module in which semiconductor elements are connected to an image sensor chip allows for, for example, reducing the length of a rigid portion at a distal end of an insertion section of the endoscope.

### SUMMARY

In accordance with one of some aspect, there is provided an endoscope including: an image sensor having a light receiving plane; a laminate provided to be opposed to an opposite side of the image sensor from the light receiving plane and having a plurality of layers formed by lamination of a plurality of semiconductor elements; and an insertion section having the image sensor and the laminate therein. The laminate includes: a first active layer in which a first active element is provided; a first passive layer in which a first passive element is provided and which is provided between the first active layer and the image sensor; and a through-silicon via provided in each of the first active layer and the first passive layer.

In accordance with one of some aspect, there is provided an imaging module including: an image sensor having a light receiving plane; and a laminate provided to be opposed to an opposite side of the image sensor from the light receiving plane and having a plurality of layers formed by lamination of a plurality of semiconductor elements. The laminate includes: a first active layer in which a first active element is provided; a first passive layer in which a first passive element is provided and which is provided between the first active layer and the image sensor; and a through-silicon via provided in each of the first active layer and the first passive layer.

### BRIEF DESCRIPTION OF THE DRAWINGS

FIG. 1 illustrates an example configuration of an imaging module.

FIG. 2 illustrates a detailed example configuration of an image sensor and a laminate.

FIGS. 3A and 3B illustrate a process for manufacturing the imaging module in the form of a wafer-level laminate.

FIG. 4 is a block diagram of the imaging module.

FIG. 5 illustrates an example configuration of an endoscope system including an endoscope.

FIG. 6 illustrates a sectional configuration of a first active layer including a signal separation circuit.

FIG. 7 is a block diagram of the imaging module.

FIG. 8 illustrates a detailed example configuration of the image sensor and the laminate.

FIG. 9 is a block diagram of the imaging module.

FIG. 10 illustrates a detailed example configuration of the image sensor and the laminate.

FIG. 11 illustrates a detailed example configuration of the image sensor and the laminate.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Exemplary embodiments are described below. Note that the following exemplary embodiments do not in any way limit the scope of the content defined by the claims laid out herein. Note also that all of the elements described in the present embodiment should not necessarily be taken as essential elements.

### 1. First Embodiment

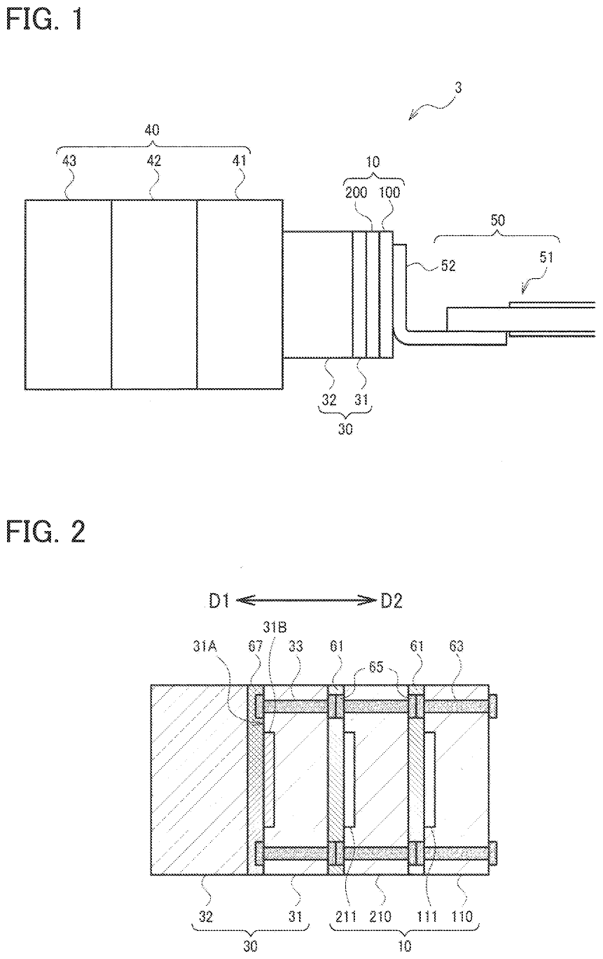

FIG. 1 is a sectional view illustrating a configuration of an imaging module 3 of the present embodiment. More specifically, FIG. 1 is a sectional view taken along a plane that is perpendicular to a substrate surface of an image sensor chip 31 (described below) or the like and parallel to an optical axis of the imaging module 3. The imaging module 3 includes an image sensor 30 and a laminate 10. The imaging module 3 may also include other components such as an optical module section 40 and a cable section 50. The imaging module 3 is contained in a housing (not shown). For example, this housing is of a cylindrical shape having a circular cross-section in the direction perpendicular to the optical axis.

It should be noted that the drawings based on respective embodiments discussed in the following description are schematic in nature, and a relationship between thickness and width of each portion, thickness ratios between respective portions, their relative angles, and other details differ from actual ones. Also, dimensional relationships and ratios between particular portions in the figures may differ from one figure to another. Additionally, certain components may be omitted from the figures.

The optical module section 40 includes a plurality of optical members 41, 42, 43. The optical member as used herein is specifically a lens. The number of optical members included in the optical module section 40 is not limited to three, and any of various numbers of optical members may be employed.

The image sensor 30 includes an image sensor chip 31 and a cover glass 32. The image sensor chip 31 includes a semiconductor, for example. As shown in FIG. 2, a light receiving section 31B is formed in a light receiving plane 31A of the image sensor chip 31. The light receiving section 31B includes, for example, a charge-coupled device (CCD) image sensor or a complementary metal-oxide-semiconductor (CMOS) image sensor. The cover glass 32 is provided on the light receiving plane 31A of the image sensor chip 31. The laminate 10 is provided on an opposite side of the image sensor 30 from the light receiving plane 31A. It should be noted that the configuration of the image sensor 30 is not limited to the above and may be modified in various ways. For example, the cover glass 32 is not an essential component of the image sensor 30.

The laminate 10 includes a plurality of semiconductor elements. The semiconductor elements as used herein are semiconductor chips fabricated by cutting a semiconductor wafer. Each semiconductor element is provided with at least one of an active element and a passive element. The active element performs active operations, such as amplification and rectification, based on electric power supplied thereto. Examples of the active element include transistors and diodes. The passive element does not perform these active operations. Examples of the passive element include resistors, capacitors, and inductors.

One semiconductor element includes a first surface provided with at least one of the active element and the passive element, a second surface on the back side of the first surface, and side surfaces connecting the first and second surfaces. More specifically, the first and second surfaces have a rectangular shape in a plan view, and the semiconductor element is generally a cuboid formed of the above first and second surfaces and four side surfaces. The plan view as referred to herein is a view of the semiconductor element when looked at in a normal direction of the first surface or the second surface. Here, the first and second surfaces correspond to substrate surfaces of a semiconductor substrate and have larger areas than the side surfaces. Hereinafter, the first and second surfaces are also referred to as primary surfaces. Likewise, the light receiving plane 31A of the image sensor chip 31 and the side thereof opposite the light receiving plane 31A are referred to as primary surfaces of the image sensor chip 31.

The laminate 10 includes a plurality of semiconductor elements laminated in a lamination direction that is along the normal direction of the primary surfaces of each semiconductor element. The lamination direction as referred to herein may be considered a direction of the optical axis of the optical module section 40. The laminate 10 includes a plurality of layers, and one semiconductor element corresponds to one layer. As shown in FIG. 1, the laminate 10 has an active layer 100 including an active element and a passive layer 200 including a passive element. The active layer 100 is a layer that constitutes the laminate 10 and is provided with an active element. More specifically, the active layer 100 is a semiconductor element provided with an active element. The passive layer 200 is a layer that constitutes the laminate 10 and is provided with a passive element. More specifically, the passive layer 200 is a semiconductor element provided with a passive element.

The cable section 50 is provided in contact with an opposite side of the laminate 10 from the image sensor 30. In the example shown in FIG. 1, the cable section 50 includes a signal cable 51 and a flexible printed circuit (FPC) board 52. The signal cable 51 is connected to the laminate 10 via the FPC board 52. The cable section 50 may, however, be modified in various ways. For example, the signal cable 51 may be directly connected to the laminate 10.

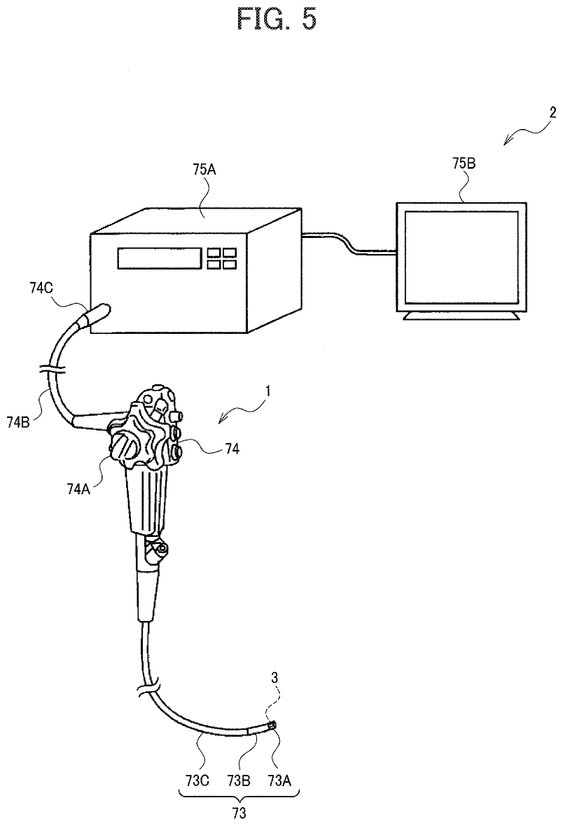

An object image formed by the plurality of optical members 41, 42, 43 included in the optical module section 40 is subjected to photoelectric conversion by the image sensor 30 provided at an image formation position on the optical module section 40 to be converted into an image signal. The image signal is output via the laminate 10, the FPC board 52, and the signal cable 51. In the case of an endoscope system 2, which is described below with reference to FIG. 5, the signal cable 51 is extended to a connector 74C via a universal cord 74B.

FIG. 2 is a schematic diagram illustrating a detailed configuration of the imaging module 3. As shown in FIG. 2, the imaging module 3 includes a first active layer 110, a first passive layer 210, and the image sensor 30 having the light receiving section 31B in the light receiving plane 31A. In the example of FIG. 2, the active layer 100 includes the single first active layer 110, and the passive layer 200 includes the single first passive layer 210.

The cover glass 32 is bonded to the light receiving plane 31A of the image sensor 30 using a transparent adhesive 67. In the following description, a direction that is perpendicular to the primary surface of the image sensor 30 and goes from the back side of the light receiving plane 31A to the light receiving plane 31A is denoted as D1, and a direction that is opposite D1 is denoted as D2. As shown in FIG. 1, the optical module section 40 is provided on the D1 side relative to the image sensor 30, and an object image passing through the optical module section 40 is formed on the light receiving plane 31A of the image sensor 30. As shown in FIG. 2, the first active layer 110, the first passive layer 210, and the image sensor 30 are arranged in this order along the direction denoted by D1.

The first active layer 110 and the first passive layer 210 are laminated via an encapsulating resin layer 61. Each of the first active layer 110 and the first passive layer 210 is formed with through-silicon vias (TSVs) 63. The first active layer 110 and the first passive layer 210 are connected to an adjacent layer in the laminate 10 or the image sensor 30 via the respective TSVs 63 and bumps 65. In the example shown in FIG. 2, the first passive layer 210 and the image sensor 30 are connected, and the first active layer 110 and the first passive layer 210 are connected.

The first active layer 110 is provided with the first active element 111. The first passive layer 210 is provided with the first passive element 211. In FIG. 2, the first active element 111 is provided in the D1-side (image sensor 30 side) one of the two primary surfaces of the first active layer 110 intersecting the lamination direction. The first passive element 211 is provided in the D1-side surface of the first passive layer 210. However, the first active element 111 may be provided in the D2-side surface of the first active layer 110 or may be provided in both primary surfaces of the first active layer 110. Similarly, the first passive element 211 may be provided in the D2-side surface of the first passive layer 210 or may be provided in both primary surfaces of the first passive layer 210.

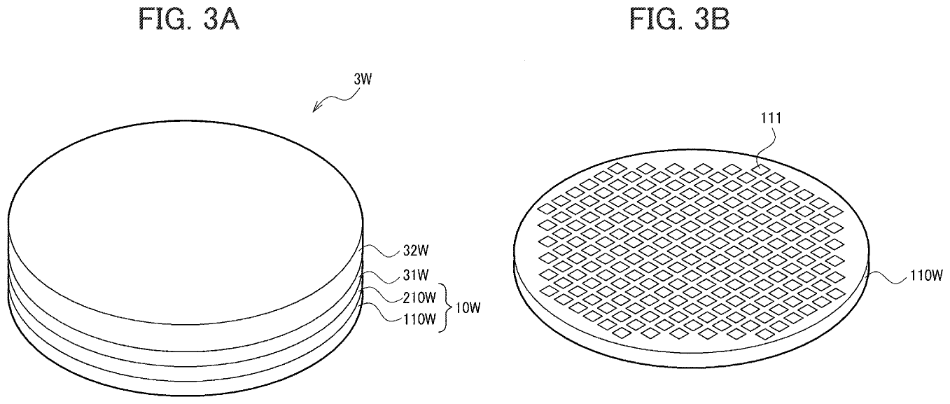

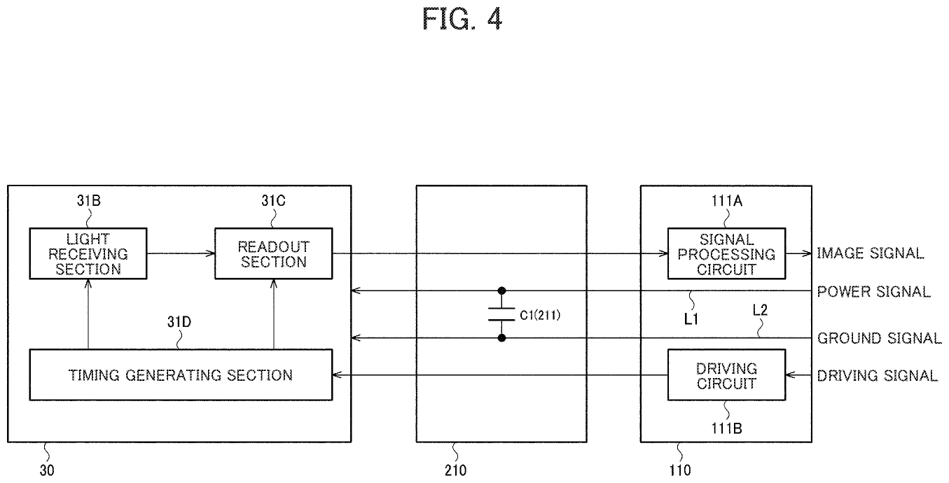

FIGS. 3A and 3B are schematic diagrams illustrating an example process for manufacturing the imaging module 3. As shown in FIG. 3A, the imaging module 3 of the present embodiment may be a wafer-level laminate fabricated by cutting a laminated wafer 3W. The laminated wafer 3W is formed by lamination of a cover glass wafer 32W, an imaging wafer 31W, and a semiconductor laminated wafer 10W. The semiconductor laminated wafer 10W is formed by lamination of semiconductor wafers 110W, 210W.

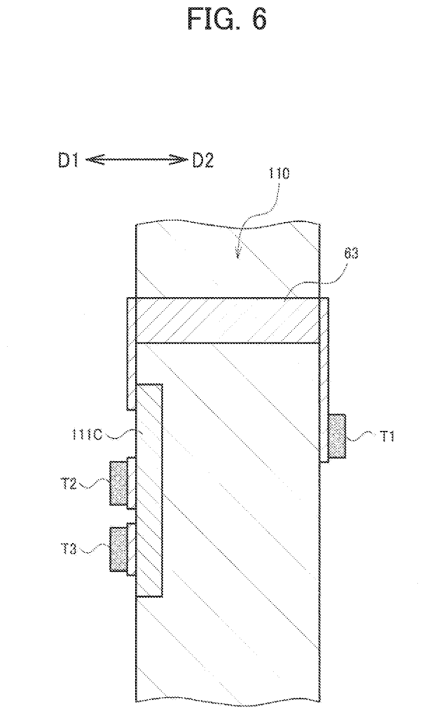

As shown in FIG. 3B, the semiconductor wafer 110W includes a plurality of first active elements 111. More specifically, the semiconductor wafer 110W is formed with a plurality of circuits each having the first active element 111. As described below, the circuit having the first active element 111 may be a signal processing circuit 111A, a driving circuit 111B (see FIG. 4), a signal separation circuit 111C (see FIG. 6), or a combination of two or more thereof. In other words, the semiconductor wafer 110W includes a plurality of semiconductor elements constituting the first active layer 110 after cutting of the wafer. For example, the first active element III is formed by a so-called semiconductor manufacturing process. Similarly, the semiconductor wafer 210W includes a plurality of semiconductor elements corresponding to the first passive layer 210. The imaging wafer 31W includes a plurality of semiconductor elements constituting the image sensor chip 31 after cutting of the wafer. A plurality of singulated imaging modules 3 are produced by cutting the laminated wafer 3W.

For example, the optical module sections 40 are disposed on the laminated wafer 3W shown in FIG. 3A and then they are cut, resulting in fabrication of the imaging modules 3 each including the optical module section 40. Alternatively, the laminated wafer 3W may include an optical member laminated wafer (not shown). For example, the optical member laminated wafer is formed by lamination of three wafers each including a plurality of optical members 41, 42, 43. Cutting the laminated wafer 3W including the optical member laminated wafer results in fabrication of single wafer-level laminates of the imaging modules 3 each incorporating the optical module section 40. The method for fabricating the imaging module 3 according to the present embodiment may be modified in various other ways.

Specific examples of the active element and the passive element are now described. The first active element 111 of the present embodiment is an element included in a peripheral circuit for, for example, driving and controlling the image sensor 30. The first passive element 211 is an element used to drive the image sensor 30, for example.

FIG. 4 is a functional block diagram of the imaging module 3 of the present embodiment. As shown in FIG. 4, the image sensor 30 includes the light receiving section 31B, a readout section 31C, and a timing generating section 31D. The first passive layer 210 includes a capacitor C1 as the first passive element 211. The first active layer 110 includes the signal processing circuit 111A and the driving circuit 111B each having the first active element 111. However, the configuration of the imaging module 3 is not limited to that shown in FIG. 4, and various modifications may be made, such as omitting some of these components and adding additional components.

The first active layer 110 of the laminate 10 is fed with power signals, ground signals, and driving signals via the signal cable 51. The driving circuit 111B of the first active layer 110 receives the driving signals and performs buffering and other processing. The driving circuit 111B outputs the processed driving signals to the timing generating section 31D of the image sensor 30. Specifically, the driving signals are transmitted to the image sensor 30 through the TSVs 63 provided in the first passive layer 210, as mentioned above with reference to FIG. 2.

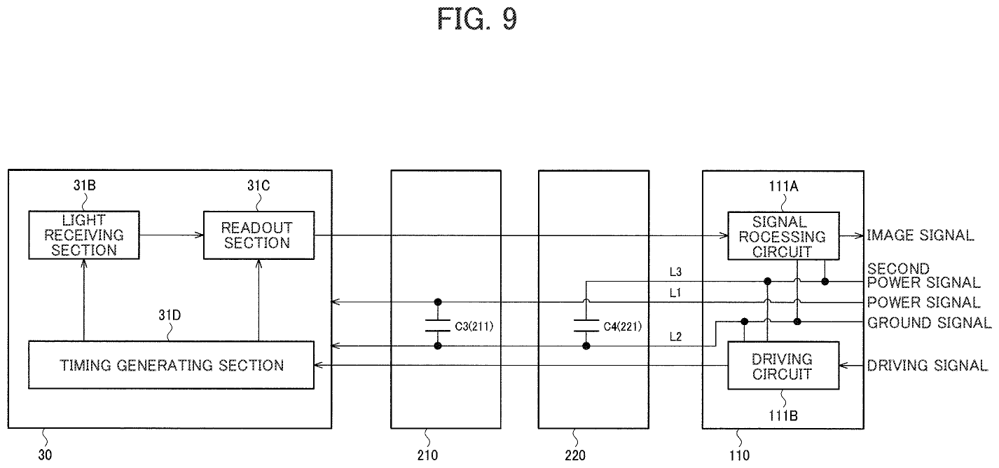

The first active layer 110, the first passive layer 210, and the image sensor 30 are provided with a power supply interconnect L1 for supplying power signals to the image sensor 30. Also, the first active layer 110, the first passive layer 210, and the image sensor 30 are provided with a ground interconnect L2 for supplying ground signals. Power signals are specifically DC signals. Each component of the image sensor 30 operates with the power signals as a power source. Here, the first passive layer 210 includes the capacitor C1 provided between the power supply interconnect L1 and the ground interconnect L2. The capacitor C1 is a bypass capacitor. Providing the bypass capacitor can reduce fluctuations in the power supply through the power supply interconnect L1. These power signals and ground signals may be used for operation of the first active element 111 included in the first active layer 110. For example, the signal processing circuit 111A is connected to the power supply interconnect L1 and the ground interconnect L2 and operates based on the power signals and the ground signals. Similarly, the driving circuit 1111B is connected to the power supply interconnect L1 and the ground interconnect L2 and operates based on the power signals and the ground signals. However, the present disclosure does not preclude a scenario where the first active element 111 operates based on different signals. For example, as described below with reference to FIG. 9, the first active element 111 may operate based on second power signals different from the power signals for the image sensor 30.

The light receiving section 31B has pixel circuits arranged in a two-dimensional array pattern. Each pixel circuit includes a photoelectric conversion element and a plurality of transistors such as transfer transistors and selection transistors.

The timing generating section 31D controls operation timings of the light receiving section 31B and the readout section 31C based on the driving signals received from the driving circuit 111B. For example, the driving signals include vertical and horizontal synchronization signals, and the timing generating section 31D generates and outputs timing pulse signals based on the driving signals.

The readout section 31C includes, for example, a vertical scanning circuit and a transfer circuit. The vertical scanning circuit controls turning on and off of the transistors in the pixel circuits based on the timing pulse signals. The transfer circuit receives pixel signals from selected pixels and outputs the pixel signals to the signal processing circuit 111A. As mentioned above, the light receiving section 31B includes the plurality of pixel circuits, so that the readout section 31C sequentially receives and outputs a plurality of pixel signals. Hereinafter, a collection of pixel signals is referred to as an image signal. It should be noted that the image sensor 30 of the present embodiment may be a CCD image sensor, a CMOS image sensor, or any other sensor using a different technology. Accordingly, the specific configuration and operation of the image sensor 30 including the timing generating section 31D and the readout section 31C may be modified in various ways.

The signal processing circuit 111A processes image signals transmitted from the readout section 31C. For example, the signal processing circuit 111A performs noise reduction processing and A/D conversion processing on the image signals. However, the A/D conversion processing may be performed by the image sensor 30, and the signal processing circuit 111A may be a circuit to perform digital noise reduction processing or the like. The signal processing circuit 111A outputs the processed image signals to the signal cable 51. In a narrow sense, the image signals having undergone the processing by the signal processing circuit 111A are image data which are digital signals. Alternatively, the signal processing circuit 111A may be a buffer circuit to perform impedance conversion processing for transmitting the image signals to the signal cable.

The technique of the present embodiment can be applied to an endoscope 1 including the above imaging module 3. FIG. 5 illustrates an example configuration of an endoscope system 2 including the endoscope 1 of the present embodiment.

As shown in FIG. 5, the endoscope system 2 includes the endoscope 1 of the present embodiment, a processor 75A, and a monitor 75B. Insertion of an elongated insertion section 73 into a body cavity of a subject allows the endoscope 1 to capture intracorporeal images and output image signals.

The endoscope 1 includes the insertion section 73, a handle section 74 disposed at a proximal end of the insertion section 73, a universal cord 74B extended from the handle section 74, and a connector 74C disposed at a proximal end of the universal cord 74B. The insertion section 73 includes a rigid distal end portion 73A in which the imaging module 3 is disposed, a bending portion 73B extended from a proximal end of the distal end portion 73A and capable of being bent for changing a direction of the distal end portion 73A, and a flexible portion 73C extended from a proximal end of the bending portion 73B. The endoscope 1 is a flexible scope, but may be a rigid scope. This means that the flexible portion and other relevant portions are not essential components. The handle section 74 is provided with a pivoting angle knob 74A as an operation portion with which a surgeon operates the bending portion 73B.

The universal cord 74B is connected to the processor 75A via the connector 74C. The processor 75A controls the entire endoscope system 2. The processor 75A also performs signal processing on image signals output from the imaging module 3 and output processing results. The monitor 75B displays the image signals output from the processor 75A as endoscope images.

The distal end portion 73A of the endoscope 1 includes a housing containing the above imaging module 3. The housing is of a cylindrical shape having a circular cross-section in the direction intersecting the optical axis. The housing, which is made of a rigid material such as metals (e.g., stainless steel), is filled with an encapsulating resin such as a silicone resin or an epoxy resin. An outer surface of the housing may be covered with a resin layer. The distal end portion 73A has round-chamfered corners. Preferably, the material of the housing has light shielding characteristics. Using such a light shielding material for the housing can inhibit effects of light on the light receiving section 31B that may enter from the side surface of the imaging module 3.

As mentioned above, the imaging module 3 of the present embodiment includes the image sensor 30 having the light receiving plane 31A and the laminate 10 provided in confronting relation to the opposite side of the image sensor 30 from the light receiving plane 31A and having the plurality of layers formed by lamination of the plurality of semiconductor elements. The laminate 10 includes the first active layer 110 provided with the first active element 111 and the first passive layer 210 provided with the first passive element 211 and positioned between the first active layer 110 and the image sensor 30. The term “between” as used herein in relation to the three layers of the first active layer 110, the first passive layer 210, and the image sensor 30 is intended to merely mean that the three layers are arranged in this order in the lamination direction, so that they may have any relationship with respect to other layers. That is, the term “between” as used herein not only encompasses configurations where the three layers are in directly adjacent relation to each other but also configurations where one or more other layers are provided between the first active layer 110 and the first passive layer 210 and/or one or more other layers are provided between the first passive layer 210 and the image sensor 30.

Here, the first active layer 110 is a semiconductor element that includes an active element, and the present disclosure does not preclude a scenario where the first active layer 110 also includes a passive element. This is the same for other active layers such as a second active layer 120, which is detailed below with reference to FIG. 11. On the other hand, the first passive layer 210 is a semiconductor element that includes a passive element and does not include an active element. However, the present disclosure does not preclude a scenario where the first passive layer 210 includes an active element that does not perform any active operation during actual operations. For example, the first passive layer 210 may include an active element that operates only during testing. This is the same for other passive layers such as a second passive layer 220 and a third passive layer 230 described below.

In the imaging module 3, the image sensor 30 and the active element generate more heat than the passive element. For this reason, if the image sensor 30 and the active element are close in distance to each other, heat generated in one of the two components may be transferred to the other, which may give rise to noise or characteristic defects. The noise as referred to herein includes, for example, isolated point noise such as white spots.

In this regard, according to the technique of the present embodiment, the first passive layer 210 is positioned between the image sensor 30 and the first active layer 110 within the imaging module 3 including the laminate 10 formed by lamination of the semiconductor elements. In other words, this effective configuration, where the laminate 10 is employed to reduce the size of the imaging module 3 while considering the layer sequence of the laminate 10, can inhibit thermal effects on the image sensor 30 which may otherwise by caused by the heat generation of the laminate 10. Specifically, the above configuration, where the image sensor chip 31, the active element chip as the semiconductor element constituting the active layer 100, and the passive element chip as the semiconductor element constituting the passive layer 200 are laminated and connected such that the passive element chip is held between the image sensor chip 31 and the active element chip, provides less heat transfer from the image sensor chip 31 to the active element chip and also provides less heat transfer from the active element chip to the image sensor chip 31. Consequently, this can inhibit noise or characteristic defects which may otherwise be caused by the thermal effects.

Additionally, positioning the passive element chip between the image sensor chip 31 and the active element chip can also reduce electromagnetic interference noise between the image sensor chip and the active element chip.

Also, as shown in FIG. 4, the first active layer 110 is provided with at least one of the driving circuit 111B and the signal processing circuit 111A each having the first active element 111. The driving circuit 111B outputs driving signals used for driving the image sensor 30. The signal processing circuit 111A processes image signals output from the image sensor 30. It should be noted that the first active layer 110 does not necessarily need to include both the driving circuit 111B and the signal processing circuit 111A. and either one may be eliminated.

The above configuration allows for enclosing the circuits for driving and controlling the image sensor 30 within the imaging module 3 while inhibiting the thermal effects from the circuits.

The first passive element 211 may further include a bypass capacitor provided between a high-side power supply interconnect for supplying high-side power signals to the image sensor 30 and a low-side power supply interconnect for supplying low-side power signals to the image sensor 30. The high-side power supply interconnect may also be used to supply high-side power signals to components other than the image sensor 30, and the low-side power supply interconnect may also be used to supply low-side power signals to components other than the image sensor 30. The high-side power signals as referred to herein are the power signals in FIG. 4, and the high-side power supply interconnect as referred to herein is the power supply interconnect L1 in FIG. 4. The low-side power signals are specifically the ground signals, and the low-side power supply interconnect is the ground interconnect L2 in FIG. 4. The bypass capacitor is provided between the power supply interconnect L1 and the ground interconnect L2.

This configuration can inhibit variations in power supply voltages supplied to the image sensor 30. Although not shown in FIG. 4, common power signals may be supplied to the image sensor 30 and the first active element 111. In this case, the capacitor, which is, e.g., a bypass capacitor, is C1 shown in FIG. 4, and a single bypass capacitor may be used to inhibit voltage variations in the common power supply. Alternatively, two bypass capacitors may be provided in the first passive layer 210, one for the image sensor 30 and the other for the first active element 111. For example, the bypass capacitor for the image sensor 30 is positioned at a location within the first passive layer 210 where a short interconnect distance is provided between that bypass capacitor and the image sensor 30. The bypass capacitor for the first active element 111 is positioned at a location within the first passive layer 210 where a short interconnect distance is provided between that bypass capacitor and the first active layer 110. As described below with reference to FIG. 9, power signals for the image sensor 30 and power signals for the first active element 111 may be different.

Additionally, as shown in FIG. 1, the imaging module 3 may further include the cable member provided in contact with the opposite side of the laminate 10 from the image sensor 30. The cable member as referred to herein is any member included in the cable section 50. In other words, the cable member provided in contact with the laminate 10 may be the FPC board 52 or the signal cable 51, or may include both of them.

This configuration allows the heat generated by the first active element 111 to be released through the cable section 50. This, in turn, can further inhibit the thermal effects on the image sensor 30.

As mentioned above with reference to FIG. 5, the technique of the present embodiment can be applied to the endoscope 1 including: the image sensor 30 including the light receiving plane 31A; the laminate 10 provided to be opposed to the opposite side of the image sensor 30 from the light receiving plane 31A and having the plurality of layers formed by lamination of the plurality of semiconductor elements; and the insertion section 73 having the image sensor 30 and the laminate 10 therein. The laminate 10 includes: the first active layer 110 in which the first active element 111 is provided; and the first passive layer 210 in which the first passive element 211 is provided and which is provided between the first active layer 110 and the image sensor 30.

Using the laminate 10, which is formed by lamination of the semiconductor elements, can make the imaging module 3 short and compact. This, in turn, can make the distal end portion 73A of the endoscope 1 short and small in diameter, allowing for reducing the invasiveness of the endoscope 1. At the same time, the heat generated by one of the image sensor 30 and the active element is inhibited from affecting the other, which leads to implementing the endoscope system 2 capable of obtaining and displaying high quality captured images with less noise.

### 2. Second Embodiment

FIG. 6 is a schematic diagram illustrating a sectional configuration of the first active layer 110 of the present embodiment. As shown in FIG. 6, the first active element 111 may include a signal separation circuit 111C. For example, a superimposed signal is input via the cable section 50 to an input terminal T1 provided on the D2-side surface of the first active layer 110. The superimposed signal as referred to herein is a signal including a superposition of the power signal and the driving signal as mentioned above with reference to FIG. 4. The input terminal as referred to herein corresponds to the bump 65, for example.

The superimposed signal input to the input terminal T1 is then input via the TSV 63 provided in the first active layer 110 to the signal separation circuit 111C provided on the D1-side surface of the first active layer 110. Here, the power signal may be a DC signal, and the driving signal may be an AC signal such as clock signals. In this case, the signal separation circuit 111C is an AC-DC separation circuit. The signal separation circuit 111C includes a capacitor provided in series with the line to which the superimposed signal is input. The drive signal, which is an AC signal, is separated through this capacitor and output from an output terminal T2. The signal separation circuit may also include an inductor provided in series with the above line. The power signal, which is a DC signal, is separated through this inductor and output from an output terminal T3. However, depending on the inductance value of the inductor and the frequency of the AC signal, the inductor may not be in a high impedance state. Accordingly, a technique is known to separate the DC signal by phase-inverting the separated AC signal with an inverting amplifier circuit and superimposing it on the input voltage. In this case, the first active element III is specifically an inverting amplifier circuit (transistor). Various other configurations of AC-DC separation circuits including active elements are known, and they can be generally used as the signal separation circuit 111C of the present embodiment. The signal separation circuit 111C of the present embodiment may be any circuit that can separate the superimposed signal into a plurality of signals, and is thus not limited to AC-DC separation circuits.

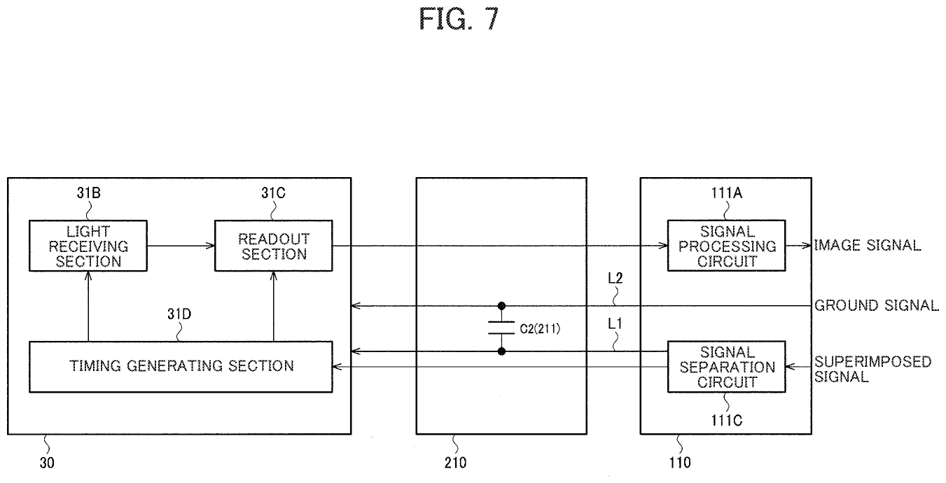

FIG. 7 is a functional block diagram of the imaging module 3 of the present embodiment. The image sensor 30 is similar to that shown in FIG. 4, so that descriptions thereof are omitted. The first passive layer 210 includes a capacitor C2 as the first passive element 211. The first active layer 110 includes the signal processing circuit 111A and the signal separation circuit 111C as the circuits each having the first active element 111. However, the configuration of the imaging module 3 is not limited to that shown in FIG. 7, and various modifications may be made, such as omitting some of these components and adding additional components. For example, power signals not shown in FIG. 7 may be supplied to the signal separation circuit 111C. The signal processing circuit 111A may operate based on power signals in common with the signal separation circuit 111C or operate based on power signals output from the signal separation circuit 111C.

As shown in FIG. 7, superimposed signals and ground signals are supplied to the first active layer 110 via the signal cable 51. The signal separation circuit 111C in the first active layer 110 receives the superimposed signals and separate them into power signals and driving signals as mentioned above. The signal separation circuit 111C outputs the separated driving signals to the timing generating section 31D of the image sensor 30. Also, the signal separation circuit 111C outputs the separated power signals to the image sensor 30 via the power supply interconnect L1 for the image sensor 30. The capacitor C2 as the first passive element 211 is provided between the power supply interconnect L1 and the ground interconnect L2. The capacitor C2 is a bypass capacitor, similarly to the capacitor C1 in FIG. 4.

In the present embodiment, too, the first passive layer 210 is provided between the image sensor 30 and the first active layer 110, similarly to the first embodiment. This arrangement can inhibit the thermal effects on the image sensor 30 which may otherwise be caused by the heat generated by the signal separation circuit 111C.

As mentioned above, the first active layer 110 may be provided with the signal separation circuit 111C having the first active element 111. The signal separation circuit 111C acquires the superimposed signals each including a superposition of the power signal supplied to the image sensor 30 and the driving signal used to drive the image sensor 30 and separates the superimposed signals into the power signals and the driving signals.

Using the signal separation circuit 111C allows for receiving and separating the superimposed signals, so that the number of cables can be reduced. For example, in the above example discussed with reference to FIG. 4, the cable section 50 is required to have at least four cables for input and output of the power signals, the ground signals, the driving signals, and the image signals. In the example shown in FIG. 7, in contrast, the cable section 50 is only required to provide input and output of the superimposed signals, the ground signals, and the image signals, so that the number of cables can be reduced compared to the example shown in FIG. 4. Thus, the size of the imaging module 3 including the cable section 50 can be reduced. Consequently, this can reduce the diameter of the endoscope 1, for example.

Additionally, using the signal separation circuit 111C allows the power signals to be output from the signal separation circuit 111C to the image sensor 30. This allows for efficient power supply by providing the output terminal T3, from which the power signals are output, on the surface of the first active layer 110 facing the image sensor 30, as shown in FIG. 6. In the present embodiment, however, the bypass capacitor is provided in the first passive layer 210 to stabilize the power supply. If the first passive layer 210 is provided on the D2 side relative to the first active layer 110, outputs from the front side (D1 side) of the active element chip need to be led to the back side (D2 side) thereof in order to connect the bypass capacitor between the power supply interconnect L1 and the ground interconnect L2. This results in the need to add the TSVs 63 and the bumps 65, leading to increasing the size of the active element chip. In other words, providing the first passive layer 210 between the image sensor 30 and the first active layer 110 can not only inhibit the thermal effects but also reduce the size of the semiconductor element including the signal separation circuit 111C.

### 3. Third Embodiment

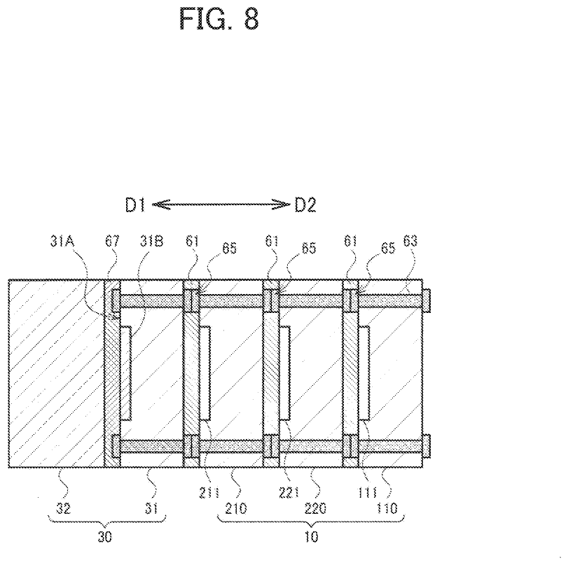

FIG. 8 is a schematic diagram illustrating a detailed configuration of the imaging module 3 of the present embodiment. As shown in FIG. 8, the imaging module 3 includes the first active layer 110, a second passive layer 220, the first passive layer 210, and the image sensor 30. In other words, the active layer 100 may include the single first active layer 110, and the passive layer 200 may include two layers of the first passive layer 210 and the second passive layer 220. As shown in FIG. 8, the first active layer 110, the second passive layer 220, the first passive layer 210, and the image sensor 30 are arranged in this order along the direction denoted by D1.

The first active layer 110, the second passive layer 220, and the first passive layer 210 are laminated via the respective encapsulating resin layers 61. Each of the first active layer 110, the second passive layer 220, and the first passive layer 210 is formed with the TSVs 63. As shown in FIG. 8, in the imaging module 3 of the present embodiment, the image sensor 30 and the first passive layer 210 are connected, the first passive layer 210 and the second passive layer 220 are connected, and the second passive layer 220 and the first active layer 110 are connected.

The first active layer 110 is provided with the first active element 111. The first passive layer 210 is provided with the first passive element 211. The second passive layer 220 is provided with a second passive element 221. As shown in FIG. 4, the circuits having the first active element 111 include the signal processing circuit 111A and the driving circuit 111B. However, the circuits having the first active element 111 may also include the signal separation circuit 111C, as mentioned above with reference to FIG. 6.

The first passive element 211 is used to drive the image sensor 30, and the second passive element 221 is used to drive the first active element 111. More specifically, the first passive element 211 is a bypass capacitor provided between the power supply for the image sensor 30 and the ground, and the second passive element 221 is a bypass capacitor provided between the power supply for the first active element 111 and the ground.

FIG. 9 is a functional block diagram of the imaging module 3 of the present embodiment. The image sensor 30 is similar to that shown in FIG. 4, so that descriptions thereof are omitted. The first passive layer 210 includes a capacitor C3 as the first passive element 211. The second passive layer 220 includes a capacitor C4 as the second passive element 221. The first active layer 110 includes the signal processing circuit 111A and the driving circuit 111B as the circuits each having the first active element 111. However, the configuration of the imaging module 3 is not limited to that shown in FIG. 9, and various modifications may be made, such as omitting some of these components and adding additional components. For example, either the signal processing circuit 111A or the driving circuit 111B may operate based on power signals for the image sensor 30. As shown in FIG. 7, the imaging module 3 may further include the signal separation circuit 111C as the first active element 111. Power signals output from the signal separation circuit 111C may be supplied to the image sensor 30 and/or the signal processing circuit 111A.

As shown in FIG. 9, the first active layer 110, the second passive layer 220, the first passive layer 210, and the image sensor 30 are provided with the power supply interconnect L1 for supplying power signals to the image sensor 30. Additionally, at least the first active layer 110 and the second passive layer 220 are provided with a second power supply interconnect L3 for supplying second power signals for the first active element 111. The first active layer 110, the second passive layer 220, the first passive layer 210, and the image sensor 30 are provided with the ground interconnect L2.

The first active layer 110 is fed with the power signals for the image sensor 30, the second power signals for the first active element 111, the ground signals, and the driving signals. The power signals input to the first active layer 110 are supplied to the image sensor 30 via the power supply interconnect L1. The second power signals are supplied via the second power supply interconnect L3 to the signal processing circuit 111A and the driving circuit 111B each having the first active element 111.

The capacitor C3 included in the first passive layer 210 is provided between the power supply interconnect L1 and the ground interconnect L2. In other words, the capacitor C3 is a bypass capacitor for inhibiting variations in the power signals for the image sensor 30. The capacitor C4 included in the second passive layer 220 is provided between the second power supply interconnect L3 and the ground interconnect L2. In other words, the capacitor C4 is a bypass capacitor for inhibiting variations in the second power signals.

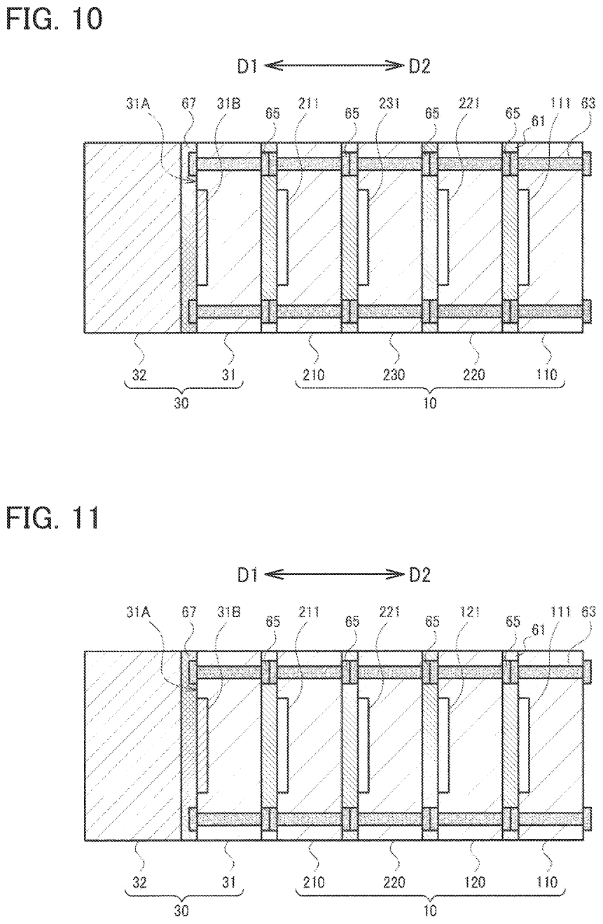

FIG. 10 is a schematic diagram illustrating another configuration of the imaging module 3. As shown in FIG. 10, the imaging module 3 includes the first active layer 110, the second passive layer 220, a third passive layer 230, the first passive layer 210, and the image sensor 30. The first active layer 110, the second passive layer 220, the third passive layer 230, the first passive layer 210, and the image sensor 30 are arranged in this order along the direction denoted by D1. The third passive layer 230 is provided with a third passive element 231 different from the first and second passive elements 211, 221. As shown in FIG. 10, the number of passive layers 200 included in the laminate 10 is not limited to one or two, and three or more passive layers 200 may be employed.

FIG. 11 is a schematic diagram illustrating still another configuration of the imaging module 3. As shown in FIG. 11, the imaging module 3 includes the first active layer 110, a second active layer 120, the second passive layer 220, the first passive layer 210, and the image sensor 30. The first active layer 110, the second active layer 120, the second passive layer 220, the first passive layer 210, and the image sensor 30 are arranged in this order along the direction denoted by D1. The second active layer 120 is provided with a second active element 121 different from the first active element 111. As shown in FIG. 11, the laminate 10 may include a plurality of active layers 100. Also, the number of active layers 100 included in the laminate 10 is not limited to one or two, and three or more active layers 100 may be employed.

As mentioned above, the laminate 10 of the present embodiment includes a semiconductor element provided with the second passive element 221 different from the first passive element 211. Here, the second passive element 221 and the first passive element 211 may be provided in a common semiconductor element. For example, both the first passive element 211 and the second passive element 221 are provided in the first passive layer 210. This configuration allows for providing a variety of passive elements in the laminate 10.

Alternatively, the laminate 10 may include the second passive layer 220 provided with the second passive element 221 and positioned between the first active laver 110 and the first passive layer 210. This configuration allows for implementing the laminate 10 including a plurality of passive layers 200 each including the passive element. Providing the plurality of passive layers 200 can increase the distance between the image sensor 30 and the first active laver 110 as compared to when the laminate 10 includes a single passive layer 200. Consequently, this can further inhibit the heat generated by one of the image sensor 30 and the active element from affecting the other.

Still alternatively, as shown in FIG. 10, the laminate 10 may include the third passive layer 230 provided with the third passive element 231 different from the first and second passive elements 211, 221 and positioned between the first passive layer 210 and the second passive layer 220. Increasing the number of passive layers 200 in this manner can further inhibit the thermal effects. It should be noted that while increasing the number of passive layers 200 offers advantages in terms of inhibiting the thermal effects, it may lead to an increase in the size of the imaging module 3 and an increase in transmission noise included in the image signals between the image sensor 30 and the signal processing circuit 111A. Accordingly, one preferably selects the number of passive layers 200 by taking into account various factors.

Additionally, as shown in FIGS. 8 and 9 in particular, the first passive element 211 is used to drive the image sensor 30, and the first passive layer 210 is positioned adjacent to the image sensor 30. The second passive element 221 is used to drive the first active element 111, and the second passive layer 220 is positioned adjacent to the first active layer 110. This arrangement can shorten the distance between the first passive element 211 and the image sensor 30 and shorten the distance between the second passive element 221 and the first active element 111.

As mentioned above, the element used to drive the image sensor 30 may be a bypass capacitor provided between the power supply interconnect L1 for the image sensor 30 and the ground interconnect L2. As mentioned above, the element used to drive the first active element 111 may be a bypass capacitor provided between the second power supply interconnect L3, which is the power supply interconnect for the first active element 111, and the ground interconnect L2. In other words, the first passive layer 210 and the second passive layer 220 each correspond to a bypass capacitor chip. In this case, the bypass capacitor chip for the image sensor 30 can be positioned closer to the image sensor chip 31, and the bypass capacitor chip for peripheral circuits can be positioned closer to the first active element 111, which is a peripheral circuit, so that the effect of inhibiting voltage variations can be maximized. In other words, this arrangement allows the corresponding bypass capacitor to be positioned near the image sensor 30 or the first active element 111, effectively reducing fluctuations in the power supply.

Still alternatively, as shown in FIG. 11, the laminate 10 may include the second active layer 120 provided with the second active element 121 different from the first active element 111 and positioned between the first active layer 110 and the first passive layer 210. For example, one of the signal processing circuit 111A and the driving circuit 111B may correspond to the first active element 111, and the other may correspond to the second active element 121. In another example, a part of the signal processing circuit 111A may correspond to the first active element 111, and another part thereof may correspond to the second active element 121. Alternatively, another active element may be added as the second active element 121. Each of these arrangements allows for providing a variety of active elements in the laminate 10 and flexibly positioning these active elements within the laminate 10. It should be noted that, from the viewpoint of inhibiting thermal effects between the image sensor 30 and the active element by providing the passive layer 200 in between, the plurality of active layers 100 may be positioned together on the D2 side of the laminate 10. For example, when the passive layer 200 includes a plurality of layers, the second active layer 120 is positioned between the first active layer 110 and one of the plurality of layers included in the passive layer 200 that is most distant from the image sensor 30.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An endoscope comprising:
an image sensor having a light receiving plane;
a laminate provided to be opposed to an opposite side of the image sensor from the light receiving plane and having a plurality of layers formed by lamination of a plurality of semiconductor elements; and
an insertion section having the image sensor and the laminate therein, wherein
the laminate includes:
a first active layer in which a first active element is provided;
a first passive layer in which a first passive element is provided and which is provided between the first active layer and the image sensor; and
a through-silicon via provided in each of the first active layer and the first passive layer.

2. The endoscope as defined in claim 1, wherein
the through-silicon via provided in the first active layer and the through-silicon via provided in the first passive layer are at identical positions in a direction perpendicular to an optical axis of the light receiving plane.

3. The endoscope as defined in claim 1, wherein
the laminate includes the semiconductor element in which a second passive element different from the first passive element is provided, and
the laminate includes a second passive layer in which the second passive element is provided and which is provided between the first active layer and the first passive layer.

4. The endoscope as defined in claim 3, wherein
the laminate includes a third passive layer in which a third passive element different from the first passive element and the second passive element is provided and which is provided between the first passive layer and the second passive layer.

5. The endoscope as defined in claim 3, wherein
the first passive element is an element used to drive the image sensor, and the first passive layer is provided adjacent to the image sensor, and
the second passive element is an element used to drive the first active element, and the second passive layer is provided adjacent to the first active layer.

6. The endoscope as defined in claim 1, wherein
the laminate includes a second active layer in which a second active element different from the first active element is provided and which is provided between the first active layer and the first passive layer.

7. The endoscope as defined in claim 1, wherein
a signal separation circuit having the first active element is provided in the first active layer, and
the signal separation circuit is a circuit to acquire a superimposed signal including a superposition of a power signal supplied to the image sensor and a driving signal used to drive the image sensor and separate the superimposed signal into the power signal and the driving signal.

8. The endoscope as defined in claim 1, wherein
at least one of a driving circuit and a signal processing circuit having the first active element is provided in the first active layer,
the driving circuit is a circuit to output a driving signal used to drive the image sensor, and
the signal processing circuit is a circuit to process an image signal output from the image sensor.

9. The endoscope as defined in claim 1, wherein
the first passive element includes a bypass capacitor provided between a high-side power supply interconnect for supplying a high-side power signal to the image sensor and a low-side power supply interconnect for supplying a low-side power signal to the image sensor.

10. The endoscope as defined in claim 1, further comprising a cable member provided in contact with an opposite side of the laminate from the image sensor.

11. An imaging module comprising:
an image sensor having a light receiving plane; and
a laminate provided to be opposed to an opposite side of the image sensor from the light receiving plane and having a plurality of layers formed by lamination of a plurality of semiconductor elements, wherein
the laminate includes:
a first active layer in which a first active element is provided;
a first passive layer in which a first passive element is provided and which is provided between the first active layer and the image sensor; and
a through-silicon via provided in each of the first active layer and the first passive layer.

12. The imaging module as defined in claim 11, wherein
the through-silicon via provided in the first active layer and the through-silicon via provided in the first passive layer are at identical positions in a direction perpendicular to an optical axis of the light receiving plane.

13. The imaging module as defined in claim 11, wherein
the laminate includes the semiconductor element in which a second passive element different from the first passive element is provided, and
the laminate includes a second passive layer in which the second passive element is provided and which is provided between the first active layer and the first passive layer.

14. The imaging module as defined in claim 13, wherein
the laminate includes a third passive layer in which a third passive element different from the first passive element and the second passive element is provided and which is provided between the first passive layer and the second passive layer.

15. The imaging module as defined in claim 13, wherein
the first passive element is an element used to drive the image sensor, and the first passive layer is provided adjacent to the image sensor, and
the second passive element is an element used to drive the first active element, and the second passive layer is provided adjacent to the first active layer.

16. The imaging module as defined in claim 11, wherein
the laminate includes a second active layer in which a second active element different from the first active element is provided and which is provided between the first active layer and the first passive layer.

17. The imaging module as defined in claim 11, wherein
a signal separation circuit having the first active element is provided in the first active layer, and
the signal separation circuit is a circuit to acquire a superimposed signal including a superposition of a power signal supplied to the image sensor and a driving signal used to drive the image sensor and separate the superimposed signal into the power signal and the driving signal.

18. The imaging module as defined in claim 11, wherein
at least one of a driving circuit and a signal processing circuit having the first active element is provided in the first active layer,
the driving circuit is a circuit to output a driving signal used to drive the image sensor, and
the signal processing circuit is a circuit to process an image signal output from the image sensor.

19. The imaging module as defined in claim 11, wherein
the first passive element includes a bypass capacitor provided between a high-side power supply interconnect for supplying a high-side power signal to the image sensor and a low-side power supply interconnect for supplying a low-side power signal to the image sensor.

20. The imaging module as defined in claim 11, further comprising a cable member provided in contact with an opposite side of the laminate from the image sensor.
